(19) United States
(12) Patent Application Publication
(10) Pub. No.: US 2017/0224879 A1
(43) Pub. Date: Aug. 10, 2017

(54) METHOD FOR MANUFACTURING A CONDUCTIVE FILM FROM AN ELECTROCHEMICAL BIOREACTOR

(71) Applicant: UNIVERSITE GRENOBLE ALPES, Saint Martin d'Heres (FR)

(72) Inventors: COSNIER Serge, Grenoble Cedex 9 (FR); HADDAD Raoudha, Grenoble Cedex 9 (FR)

(73) Assignee: UNIVERSITE GRENOBLE ALPES, Saint Martin d'Heres (FR)

(21) Appl. No.: 15/503,392

(22) PCT Filed: Jul. 30, 2015
(86) PCT No.: PCT/FR2015/052116

§ 371 Date: Feb. 11, 2017

(30) Foreign Application Priority Data
Aug. 21, 2014 (FR) 1457917

(51) Int. Cl. A61L 31/04; A61F 2/82; A61L 31/14; H01M 8/16; C23F 13/10

(52) U.S. Cl. CPC A61L 31/047; H01M 8/16; C23F 13/10; A61L 31/14; A61F 2/82; H01M 2250/30; C23F 2213/30

(57) ABSTRACT

The invention relates to a device that is to be implanted in vivo and includes a stent (46) surrounded, at least partially, by at least one flexible conductive film (54, 56) containing chains of a linear polymer, each of which has carbon nanotubes connected thereto via pi-pi interactions. Said film is functionalized by enzymatic grafting so as to form an electrochemical bioreactor element.

The present patent application claims the priority benefit of French patent application FR14/57917 which will be incorporated herein by reference.

### BACKGROUND

The present invention relates to a method of manufacturing a conductive film capable of forming an element of an electrochemical bioreactor at the level of which a reaction between elements confined in the bioreactor and compounds present in a liquid medium having the bioreactor immersed therein is capable of occurring. The reaction may for example result in a deformation of the bioreactor, in the generation of an electric potential, or in the chemical transformation of the compound interacting with the bioreactor.

A bioreactor causing the generation of an electric potential may form a bioelectrode of a biofuel cell or of a biosensor, of sugar/oxygen type, for example, glucose/oxygen.

A bioreactor causing the chemical transformation of a compound interacting with the bioreactor for example forms a glucose killer by, for example, transforming glucose into a compound which will for example be eliminated by the organism where the bioreactor is implanted.

Although the invention and the state of the art are described herein mainly in the case of bioelectrodes, it should be understood that the invention applies to any electrochemical bioreactor, and particularly to a bioreactor implantable in vivo.

### DISCUSSION OF THE RELATED ART

Various types of solid bioelectrodes are described in prior art. For example, French patent application N° 10/52657 (B10272) describes an electrode pellet obtained by compression of an electrically-conductive material such as graphite, of an enzyme, and possibly of an electrically-conductive polymer. The pellet has the shape of a disk having a thickness greater than 0.5 mm and having a diameter greater than 0.5 cm. Although such a pellet can be used as a bioelectrode, its stiffness and its bulk limit its use, particularly in body parts having small volumes, for example, in a blood vessel.

Article “Plasma functionalization of lucky paper and its composite with phenylethynyl-terminated polyimide” of Qian Jiang et al. published in February 2013 in volume 45 of Journal “Composites Part B: Engineering”, describes the manufacturing of a conductive film which is a composite of carbon nanotubes and of a polyimide.

FIGS. 1A to 1F schematically illustrate steps of the manufacturing method described in this article.

At the step shown in FIG. 1A, carbon nanotubes 1 are dispersed in a solvent 3 such as methanol. At the step shown in FIG. 1B, carbon nanotubes 1 suspended in the solvent are vacuum-filtered through a membrane 5 having pores 7 having a 0.22 μm average diameter. As shown in FIG. 1C, a film 9 of carbon nanotubes is obtained at the surface of membrane 5. Film 9 is treated by means of a plasma 11 so that the nanotubes of the film become hydrophilic. At the step of FIG. 1D, a suspension 13 of a phenylethynyl-terminated polyimide 15 in a solvent 17 is prepared. At the step of FIG. 1E, suspension 13 is vacuum-filtered through film 9 resting on membrane 5. As shown in FIG. 1F, a composite film 19 comprising carbon nanotube film 9 having a surface coated with polyimides 15 is obtained.

It is here more particularly provided to associate a stent and at least one flexible conductive film of a bioreactor.

### SUMMARY

An embodiment provides a device intended to be implanted in vivo, comprising a stent at least partially surrounded with at least one flexible conductive film, comprising chains of a linear polymer having carbon nanotubes bonded by pi stacking to each of them, the film being functionalized by enzyme grafting to form an element of an electrochemical bioreactor.

According to an embodiment the flexible conductive film forms an electrochemical bioelectrode.

According to an embodiment, the device comprises two film portions, each of which is rolled around substantially half the periphery of the stent, respectively forming an anode and a cathode capable of being electrically coupled to an energy storage device.

According to an embodiment, the energy storage device is capable of being coupled to the stent and to a reference electrode.

According to an embodiment, an electrically insulating biocompatible film is interposed between the stent and the flexible conductive anode and cathode films.

According to an embodiment, the device comprises a single film portion rolled around substantially the entire periphery of the stent, respectively forming an anode capable of being electrically coupled to the stent.

### BRIEF DESCRIPTION OF THE DRAWINGS

The foregoing and other features and advantages will be discussed in detail in the following non-limiting description of specific embodiments in connection with the accompanying drawings, among which:

FIGS. 1A to 1F, previously described, schematically illustrate steps of a method of manufacturing a composite film of carbon nanotubes;

FIGS. 2A to 2C schematically illustrate steps of an embodiment of a method of manufacturing a flexible conductive film;

FIG. 3 schematically illustrates the structure of a flexible conductive film formed according to the method of FIGS. 2A to 2C;

FIG. 4 schematically illustrates the flexible conductive film of FIG. 3 functionalized by an enzyme comprising a hydrophobic site;

FIG. 5 schematically illustrates the flexible conductive film of FIG. 3 functionalized by an enzyme comprising no hydrophobic site;

FIGS. 6A and 6B are simplified cross-section views showing an embodiment of a stent coupled to a biofuel cell; and

FIGS. 7A and 7B are simplified cross-section views showing an embodiment of stent coupled to a bioanode.

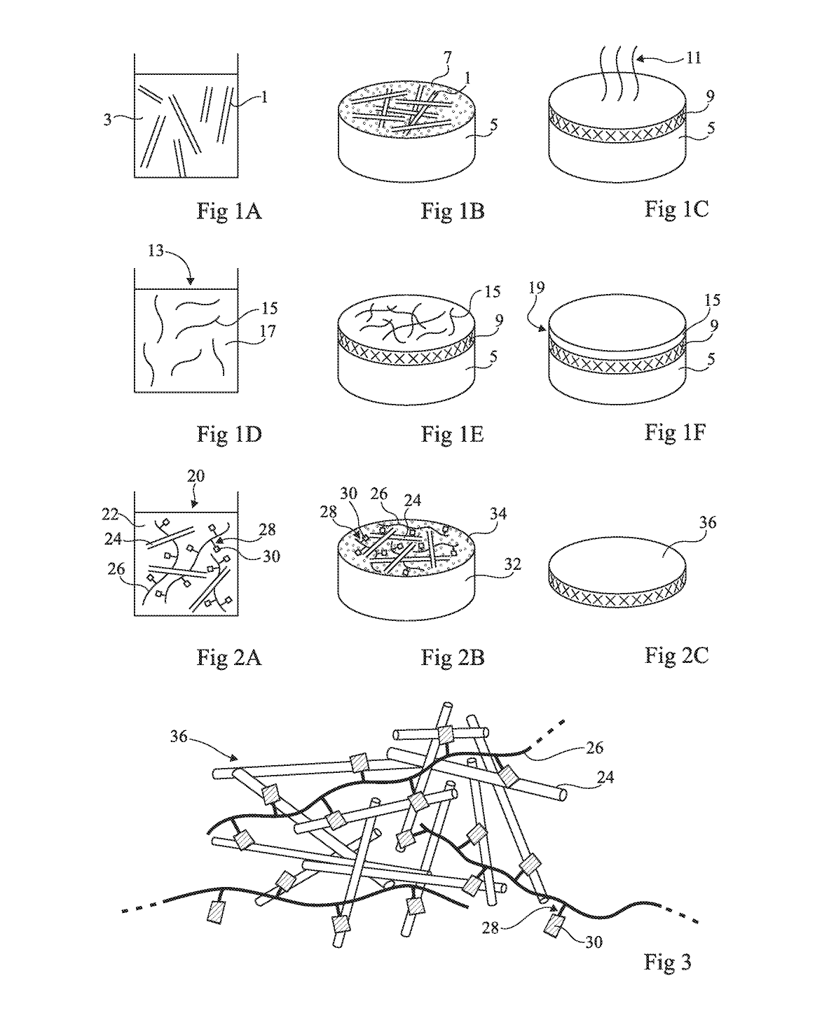

For clarity, the same elements have been designated with the same reference numerals in the various drawings and, further, the various drawings are not to scale.

### DETAILED DESCRIPTION OF THE PRESENT EMBODIMENTS

FIGS. 2A to 2C schematically illustrate successive steps of an embodiment of a flexible conductive film manufacturing mode.

At the step shown in FIG. 2A, a suspension 20 comprising, in a solvent 22, carbon nanotubes 24 and chains 26 of a linear polymer, has been prepared. Preferably, the solvent 20 is hydrophobic. The solvent may be selected from the group comprising dimethylformamide (DMF), tetrahydrofuran (THF), and chloroform. Each chain 26 of the linear polymer supports a succession of functional groups 28 comprising pi conjugated groups 30. Carbon nanotubes 24 are formed of the rolling of one or of a plurality of graphene sheets in cylinders. The cylinders are conductive due to the mobility of electrons on graphene, which comprises many pi conjugated groups. Thus, a pi conjugated group 30 of a chain 26 of the linear polymer may bond by pi stacking to a pi conjugated group of a carbon nanotube 24.

Carbon nanotubes 24 are single-walled or multi-walled nanotubes and may have a length in the range from 100 nm to 5 μm. Each functional group 28 comprising a pi conjugated group 30 is for example a macrocycle such as porphyrins and phthalocyanine, or an aromatic compound such as pyrene, benzene, indole, azulene, phenothiazines, or naphthalene. The linear polymer may be selected from the group comprising poly-norbornenes. polyvinylpyrrolidone (PVP), and sodium polystyrene sultanate (PSS). Preferably, the distance between two successive pi conjugated groups 30 of a same chain 26 is shorter than the length of carbon nanotubes 24. This distance is for example in the range from 5 to 50 nm for nanotubes having a length from 200 to 500 nm. The length of chains 26 of the linear polymer is selected to support a plurality of functional groups 28, for example, at least three functional groups 28, and preferably at least fifty functional groups 28. The length of a chain may be greater than 0.1 μm, preferably greater than 10 μm. The weight of the assembly of carbon nanotubes 24 in suspension 20 is for example from two to ten times greater than the weight of the assembly of chains 26 of the linear polymer.

At the step illustrated in FIG. 2B, suspension 20 is vacuum-filtered through a membrane 32, for example a PTFE (PolyTetraFluoroEthylene) membrane, comprising pores 34 having a diameter for example in the range from 0.1 to 0.5 μm. Chains 26 of the linear polymer having carbon nanotubes bonded thereto 24 then accumulate in a film at the surface of membrane 32.

As illustrated in FIG. 2C, after the film has been separated from membrane 32, a film 36 comprising carbon nanotubes bonded to the linear polymer chains is obtained. As an example, the thickness of film 36 is in the range from 0.01 to 1 mm. The surface concentration of carbon nanotubes may be 3.4 mg/cm² and that of the linear polymer chains may be 0.56 mg/cm².

FIG. 3 is an illustration of chains 26 of linear polymer bonded to carbon nanotubes 24 in film 36. Carbon nanotubes 24 are bonded by pi stacking with pi conjugated groups 30 of functional groups 28 supported by chains 26 of the linear polymer. A chain 26 supports a plurality of nanotubes 24 and each nanotube may be bonded to a plurality of chains.

Carbon nanotubes 24 of film 36 are in contact with one another, whereby film 36 is electrically conductive. Due to the fact that chains 26 of the linear polymer can deform under the effect of mechanical stress, the obtained film 36 is flexible. In particular, the inventors have observed that such a flexible conductive film may be rolled on itself without breaking.

To form from flexible conductive film 36 of FIG. 3 an element of an implantable bioreactor, it is here provided to functionalize the film, for example, by grafting enzymes and possibly redox mediators thereto.

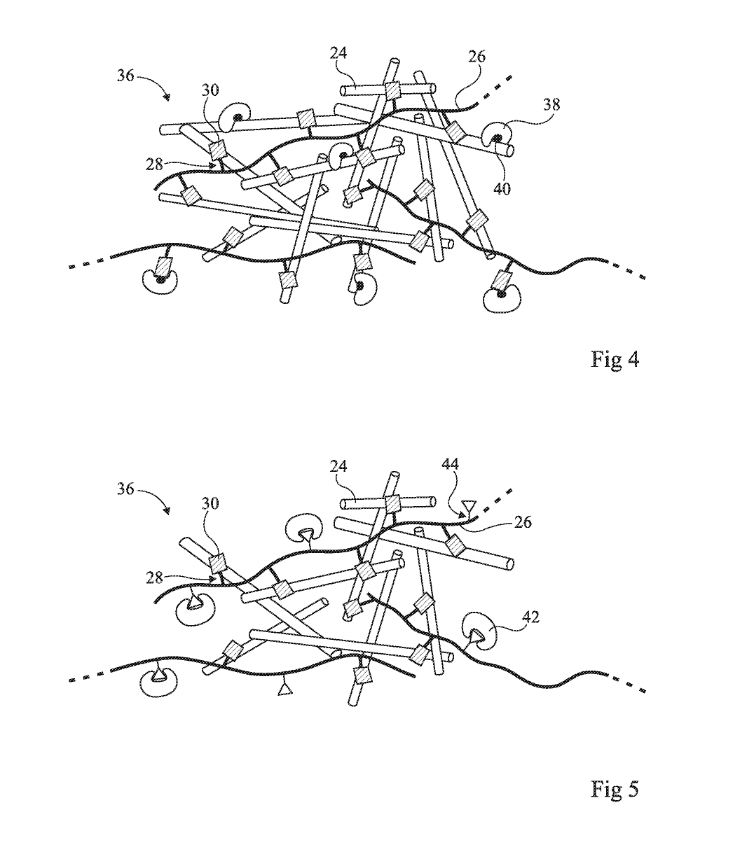

FIG. 4 is an illustration of flexible conductive film 36 of FIG. 3 functionalized by an enzyme 38, for example, a laccase, comprising a hydrophobic site 40. The hydrophobic site 40 of each enzyme 38 adsorbs on the hydrophobic surface of carbon nanotubes 24, whereby the enzyme is immobilized on the nanotubes. Hydrophobic site 40 of enzyme 38 may also bond to a pi conjugated group 30 of a functional group 28 of a chain 26. As a result, a large quantity of enzymes 38 may be grafted to flexible conductive film 36.

In order for enzymes 38 to graft to film 36, the latter is for example immersed in a solvent, preferably water, comprising suspended enzymes 38. As a variation, a suspension comprising enzymes 38 may be poured on film 36. The suspension may also be made to cross the film.

FIG. 5 is an illustration of flexible conductive film 36 of FIG. 3 functionalized by an enzyme 42 which does not comprise a hydrophobic site. In this case, flexible conductive film 36 for example comprises functional groups 44 capable of bonding to enzyme 42. In the shown embodiment, chains 26 of the linear polymer support functional groups 44 in addition to functional groups 28 comprising pi conjugated groups 30. Thus, at least some of the functional groups 44 may have one or a plurality of enzymes 42, which bond to the flexible conductive film via a specific interaction with functional group 44. bonded thereto. Similarly to what has, been described in relation with FIG. 4, enzyme 42 is grafted to the film, for example, by wetting of the latter with a suspension comprising the enzyme.

As an example, in the case where the enzyme is an avidin which is modified by biotin patterns, each of functional groups 44 comprises a biotin pattern.

In an alternative embodiment, it may be provided to use bifunctional molecules, each of which supports on the one hand a functional group 28 comprising a pi conjugated group 30 capable of bonding to an element of the flexible conductive film, and on the other hand a functional group 44 capable of bonding to enzyme 42. In this case, the film is for example wet by a suspension comprising the bifunctional molecules before the enzyme is grafted to the film.

In the same way as flexible conductive film 36 of FIG. 3 has been specialized with functional groups 44 capable of bonding to an enzyme 42. film 36 may be specialized with functional groups capable of bonding to a redox mediator. In this case, it is provided chains 26 of the linear polymer to support functional groups capable of bonding to the redox mediator in addition to functional groups 28 and to possible functional groups 44. It may also be provided for the functional groups capable of bonding to the redox mediator to be supported by bifunctional molecules, or for the redox mediator to be functionalized by a functional group capable of directly bonding to carbon nanotubes 24. The film specialized by the functional groups capable of bonding to the mediator is then wet with a suspension comprising the mediator, which grafts to the film

In alternative embodiments, it may also be provided for chains 26 of the linear polymer to be initially functionalized by the redox mediator.

As an example, in the case where the redox mediator is toluidine blue, or trimethylthionine hydrochloride, it is provided for the film to comprise functional groups comprising activated esters such as N-Hydroxysuccinimide reacting with the amino pattern of toluidine blue. The redox mediator inlay also be viologen, in this case, it is provided to functionalize the viologen with a pi conjugated group such as a pyrene, the viologen will then be bonded to the film by pi stacking between a carbon nanotube and the pi conjugated group of the functionalized viologen.

The embodiments described in relation with FIGS. 4 and 5 enable to obtain bioreactors, for example, bioelectrodes, with a flexible conductive film 36 functionalized by an enzyme and a possible redox mediator. The enzyme and the possible redox mediator are linked to functional groups of the film, which advantageously prevents their dispersion in the medium where the bioreactor is immersed.

### Applications

A flexible conductive film 36 may be used without having been functionalized by an enzyme and a possible redox mediator. Such a conductive film may for example be used for the manufacturing of flexible photovoltaic cells. Such a flexible photovoltaic cell may comprise film 36 which forms a flexible support and a hole collector, an active layer, for example, a mixture of polymers, deposited on support film 36, and a transparent conductive layer, for example, silver nanotubes, deposited on the active layer and enabling to let through photons which play the role of electron collectors.

A biofuel cell may be formed from flexible conductive films 36 associated with enzymes and with possible redox mediators. In the case of a biofuel cell of sugar-oxygen type, the anode enzyme is capable of catalyzing the oxidation of a sugar, the cathode enzyme is capable of catalyzing the reduction of oxygen, the possible redox mediator of the anode has a low redox potential capable of exchanging electrons with the anode enzyme, and the possible redox mediator of the cathode has a high redox potential capable of exchanging electrons with the cathode enzyme. When oxygen is reduced by the cathode and a sugar is oxidized by the anode, a potential difference between the bioelectrodes of the biofuel cell is obtained. The anode enzyme is for example selected from the group comprising glucose oxidase if the sugar is glucose, and lactose oxidase if the sugar is lactose, and the cathode enzyme is for example selected from the group comprising polyphenol oxidase, bilirubin oxidase, and laccase.

Such a biofuel cell with flexible bioelectrodes may be formed by stacking a flexible anode film 36 and a flexible cathode film 36 separated and electrically insulated from each other by an insulating film allowing ions to pass, for example, paper, this film being made of a biocompatible material in the case where the biofuel cell is intended to be implanted in vivo. Since conductive films 36 may have a small thickness in the order of sonic ten micrometers, the stacking of these films enabling to obtain flat biofuel cells of very small thickness. Due to the fact that anode and cathode films 36 are flexible, the forming of biofuel cells of small bulk may also be targeted by rolling such a stack on itself.

In another example of application, films 36 functionalized by enzymes and possible redox mediators are biased to promote electro-enzymatic reactions resulting in the generation or in the destruction of a substance. In this case, there may be a single enzyme promoting a single reaction, or there may be a plurality of films 36 functionalized by different enzymes, the enzymes for example having complementary activities so that each enzyme transforms the products of a reaction promoted by another enzyme.

It is here also provided to associate a stent and at least one flexible conductive film of a bioreactor such as previously described.

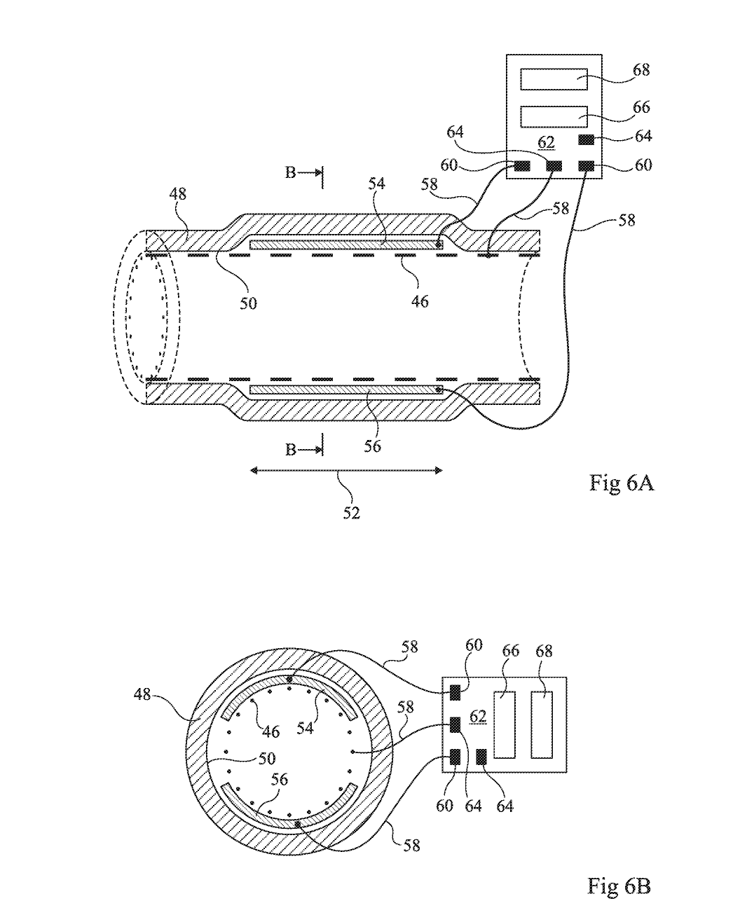

FIGS. 6A and 6B are cross-section views schematically showing a stent 46 coupled with a sugar-oxygen biofuel cell with flexible bioelectrodes, FIG. 6B being a cross-section view along plane BB of FIG. 6A.

Stent 46 made of an electrically-conductive material is arranged in a duct 48, for example, a blood vessel, so that the outer surface of the stent is pressed against inner wall 50 of duct 48. A flexible conductive cathode film 54 of a biofuel cell is interposed between the stent and the inner wall of the duct on a portion of the outer surface of the stent, for example, at the level of a central portion 52 thereof. A flexible conductive anode film 56 of the biofuel cell is interposed between the stent and the inner wall of the duct on a portion of the outer surface of the stent, for example, substantially at the surface of flexible cathode film 54. An electrically-insulating biocompatible film (not shown) is interposed between stent 46 and flexible conductive films 54 and 56, the insulating film being for example arranged on the entire outer surface of central portion 52.

A conductor 58 electrically connects flexible cathode film 54 to an input terminal 60 of an energy storage device 62 arranged outside of duct 50. Similarly, a conductor 58 electrically connects flexible anode film 56 to another input terminal 60 of storage device 62. An output terminal 64 of storage device 62 is electrically connected to stent 46 via a conductor 58. Storage device 62 further comprises another output terminal 64 electrically connected to a reference electrode, not shown, the reference electrode being for example arranged in duct 48. As an example, conductors 58 are wires or bands, for example made of platinum.

Stent 46 advantageously enables to hold in place anode and cathode films 54 and 56 against the inner wall of a duct such as a blood vessel. This is made possible due to the use of bioelectrodes with flexible conductive films 54 and 56 of the type of previously-described film 36, which take the shape of the stent and of the duct.

In operation, a potential difference is obtained between cathode film 54 and anode film 56, which enables to electrically charge a storage element 66 of device 62 such as a capacitor or a battery. An element 68 for controlling storage device 6 connects, for example, periodically, storage element 66 to output terminals 64 of device 62 to apply a potential difference between stent 46 and the reference electrode. The potential difference is selected so that the stent is at a negative potential, which advantageously prevents the accumulation of proteins at the stent surface and the oxidation of the material forming the stent. Such an association of a biofuel cell with bioelectrodes having a flexible conductive film and of a stent enables to increase the stent lifetime.

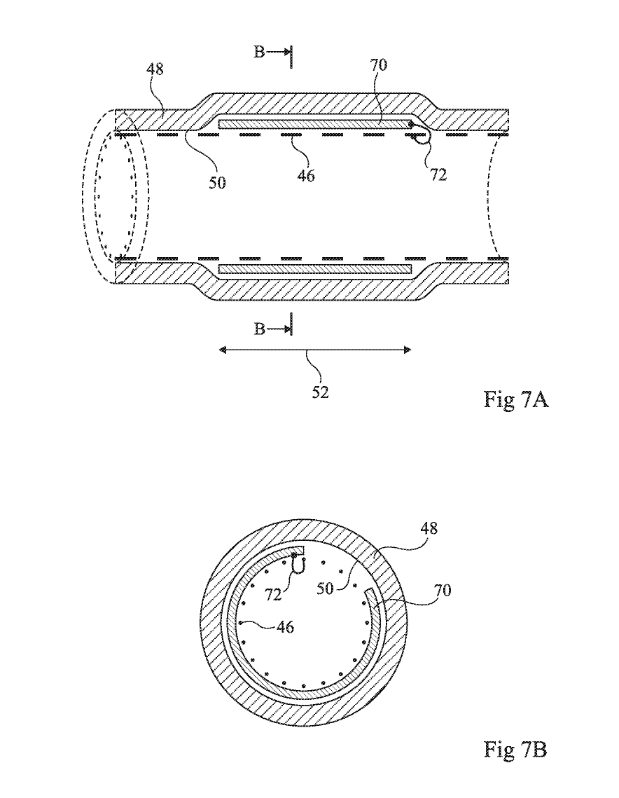

FIGS. 7A and 7B are cross-section views schematically showing a stent 46 coupled with a flexible anode film 70, FIG. 7B being a cross-section view along plane BB of FIG. 7A.

Stent 46 is arranged in a duct 48, for example, a blood vessel, so that the outer surface of the stent is pressed against inner wall 50 of duct 48. Anode film 70 is interposed between the stent and the duct on all or part of the outer surface of the stent, for example, at the level of a central portion 52 of the stent. A conductor 72, for example, a wire or a band which may be made of platinum, electrically connects film 70 to stent 46. A reference electrode, not shown, is electrically connected to the stent for example via a resistor, the reference electrode being for example arranged in the duct. In operation, flexible conductive anode film 70 negatively charges, which enables to apply a negative potential to stent 46. The application of a negative potential to stent 46 advantageously enables to prevent the accumulation of proteins at the stent surface and the oxidation of the material forming the stent.

FIGS. 6A, 6B, 7A, and 7B show a stent positioned in a duct. The diameter of the stent thus positioned is greater than the diameter of the stent before it is positioned in the duct. Before it is positioned in the duct, the retracted stent is wrapped in the flexible conductive film(s) of a bioreactor rolled around the outer surface of the stent, the flexible film(s) being held in place around the stent by a strap which can be easily broken or cut, for example, by a cord or a ring. The assembly of the stent wrapped with the flexible conductive film(s) is inserted into the duct, after which the stent material expands, causing the breakage of the strap holding the film(s) rolled around the stent. The flexible film(s) are unrolled when the stent expands until the outer surface of the stent takes the shape of the inner wall of the d ct and holds in place the flexible conductive film(s) surrounding the stent.

### Variations

Specific embodiments have been described. Various variations and modifications will occur to those skilled in the art. In particular, the previously-described flexible conductive films may be functionalized by other compounds, by other enzymes, and by other redo: mediators than those indicated as an example in the present disclosure.

Although a flexible conductive film bonded to a single enzyme and possibly to a single redox mediator has been described, more than one enzyme and/or more than one redox mediator may be bonded to a same conductive film.

In FIGS. 6A and 6B, a biofuel cell with flexible bioelectrodes held in place in a duct by a stent has been described, the biofuel cell enabling to charge a storage device arranged outside of the duct. The biofuel cell associated with the stent may also be used to power another device such as a sensor or, in the case where the stent and the biofuel cell art implanted in vivo, a pace maker. Further, although an anode film oiled around a portion of the stent circumference and a cathode film rolled around another portion of the stent circumference has been described in the drawings, it may also be provided for all or part of the stent circumference to be successively coated with an anode or cathode film, with an insulating film, and with a respective cathode or anode film.

In FIGS. 7A and 78, the bioanode is electrically connected to the stent via a conductor 72. The conductor may be suppressed, the electric connection between the stent and flexible conductive film 70 then being achieved by simple contact between these elements.

The previously-described functionalized flexible conductive films may be coated with a semi-permeable membrane to let through the reactants of the redox reaction and block other heavier elements such as chains 26 of a linear polymer, enzymes, and carbon nanotubes, its the case where the conductive films form a bioreactor intended to be implanted in vivo, the membrane is made of a biocompatible material, for example, of chitosan, or of the material designated with trade name Dacron.

Various embodiments with different variations have been described hereabove. It should be noted that those skilled in the art may combine various elements of these various embodiments without showing any inventive step. In particular, the order and the number of steps of the previously-described method may be adapted by those skilled in the art. For example, an enzyme and a redox mediator may be grafted to a film supporting groups specific to this enzyme and this mediator by the wetting of the film with a single suspension comprising the enzyme and the redox mediator.

1. A device intended to be implanted in vivo, comprising a stent at least partially surrounded with at least one flexible conductive film, comprising chains of a linear polymer having carbon nanotubes bonded by pi stacking to each of them, the film being functionalized by enzyme grafting to form an element of an electrochemical bioreactor.

2. The device of claim 1, wherein said at least one flexible conductive film forms an electrochemical bioelectrode.

3. The device of claim 2, comprising two film portions, each of which is rolled around substantially half the periphery of the stent, respectively forming an anode and a cathode capable of being electrically coupled to an energy storage device.

4. The device of claim 3, wherein the energy storage device is capable of being coupled to the stent and to a reference electrode.

5. The device of claim 3, wherein an electrically-insulating biocompatible film is interposed between the stent and the flexible conductive anode and cathode films.

6. The device of claim 2, comprising a single film portion rolled around substantially the entire periphery of the stent, respectively forming an anode capable of being electrically coupled to the stent.

7. A method of manufacturing the device of claim 1, wherein the manufacturing of the flexible conductive film comprises the successive steps of:
a) preparing a suspension comprising carbon nanotubes and chains of a linear polymer, each of said chains supporting a succession of functional groups, at least some of which comprise pi conjugated groups; and
b) vacuum filtering the first suspension to obtain a film of said chains having the nanotubes bonded thereto by pi stacking.

8. The method of claim 7, wherein, for the enzyme grafting, bifunctional molecules are used, each of which supports, on the one hand, a functional group comprising a pi conjugated stack capable of bonding to an element of the film and, on the other hand, a functional group capable of bonding to the enzyme.

9. The method of claim 7, further comprising a step of grafting redox mediators to said film.

10. The method of claim 7, wherein each of said functional groups comprising a pi conjugated group is a pyrene.

11. The method of claim 7, wherein the linear polymer is selected from the group comprising polynorbornenes, polyvinylpyrrolidone, and sodium polystyrene sulfonate.

12. The method of claim 7, wherein a distance shorter than the length of the nanotubes separates two successive pi conjugated groups of a same chain of the linear polymer.

13. The method of claim 7, wherein the length of each of said chains is greater than 0.1 μm.

14. The method of claim 7, wherein, in said suspension, the proportion by weight of the carbon nanotubes to said chains is in the range from 2 to 10.
